(19) United States
(12) Patent Application Publication
(10) Pub. No.: US 2008/0279749 A1
(43) Pub. Date: Nov. 13, 2008

(54) Method for Further Processing the Residue Obtained During the Production of Fullerene and Carbon Nanostructures

(76) Inventors: Nicolas Probst, Brussels (BE); Frederic Fabry, Le Cannet (FR); Thomas Grunberger, Brussels (BE); Eusebiu Grivei, La Hulpe (BE); Laurent Fulcheri, Mouanx-Sartoux (FR); Jose Gonzalez-Aguilar, Juan-les-Pins (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/912,471

(22) PCT Filed: Apr. 25, 2006
(86) PCT No.: PCT/EP2006/061825

§ 371 Date: Jun. 5, 2008

(30) Foreign Application Priority Data
Apr. 26, 2005 (DE) 10 2005 019 301.3

(51) Int. Cl. C01B 31/00; C07C 211/01; C07C 211/00

(52) U.S. Cl. 423/415.1; 423/414; 564/463; 564/305

(57) ABSTRACT

The present invention relates to a process for further processing of the carbon-containing residue derived from fullerene production and from carbon-nanostructures production, characterized in that the residue is functionalized via introduction of chemical substituents, and the functionalization is carried out during or after the production process. The functionalized carbon-containing residue obtainable by the process is also provided, as is its use as a hydroxylating agent, wetting agent, additive in rubber compounds, and for tether-directed remote functionalization.

### FIELD OF THE INVENTION

The present invention relates to a process for further processing of the carbon-containing residue derived from fullerene production and from carbon-nanostructures production, to the processed residue, and its use.

### BRIEF DESCRIPTION OF THE PRIOR ART

C₆₀ and C₇₀ fullerenes, which are carbon compounds having not only 6- but 5-membered rings in the form of closed cages and having an even number of carbon atoms, were first described by Kroto et al. in carbon vapour, obtained via laser irradiation of graphite (Nature 318 (1985), 162-164). Since that time, the number of known fullerenes has risen rapidly and comprises C₇₆, C₇₈, C₈₄ and larger structures, including “giant fullerenes”, characterized via Cₙ, where n=100, nanotubes and nanoparticles. Carbon nanotubes have promising applications, encompassing electronic apparatus on the nano scale, materials with high strength, electronic field emission, tips for scanning probe microscopy, and gas storage.

The following patent specifications, inter alia, describe the production of fullerenes: U.S. Pat. No. 6,358,375; U.S. Pat. No. 5,177,248; U.S. Pat. Nos. 5,227,038; 5,275,705; U.S. Pat. No. 5,985,232. There are currently five main ways of synthesizing carbon nanotubes. These include laser ablation of carbon (Thess, A. et al., Science 273 (1996), 483), electric arc discharge using a graphite rod (Journet C. et al., Nature 388 (1997), 756), chemical vapour deposition using hydrocarbons (Ivanov, V. et al., Chem. Phys. Lett. 223, 329 (1994); Li, A. et al., Science 274, 1701 (1996)), the solar process (Fields, Clark L., et al, U.S. Pat. No. 6,077,401), and plasma technology (European Patent Application EP0991590).

U.S. Pat. No. 5,578,543 describes the production of multiwall carbon nanotubes via catalytic cracking of hydrocarbons. The production of single-wall carbon nanotubes via laser techniques (Rinzler, A. G. et al, Appl. Phys. A. 67, 29 (1998)) and electric arc techniques (Haffner, J. H. et al., Chem. Phys. Lett. 296, 195 (1998)) has been described.

U.S. Pat. No. 5,985,232 relates to a process for production of fullerene nanostructures which involves combustion of an unsaturated hydrocarbon and oxygen in a combustion chamber at reduced pressure with no electric arc discharge, thus generating a flame, collection of the condensable portions of the flame, whereupon the condensable portions comprise fullerene nanostructures and carbon black, and the isolation of the fullerene nanostructures from the carbon black. The obligatory isolation of the fullerene structures from the carbon black can be carried out via known extraction and purification processes. Among these are simple and Soxhlet extraction in solvents of various polarity. The condensable portions can also be obtained via electrostatic separation processes or via inert separation processes using aerodynamic forces. Another method described as suitable for isolation and purification of the fullerene structures is HPLC. US '232 does not reveal any further processing of the carbon-containing residue produced during fullerene production.

Similar structures have been found by Donnet and collaborators using furnace blacks. However, when furnace blacks are used, these fullerene-type structures are produced only rarely and in most instances only to a very limited extent.

### BRIEF DESCRIPTION OF THE INVENTION

The present invention provides a process for further processing of the carbon-containing residue derived from fullerene production and from carbon-nanostructures production, characterized in that the residue is functionalized via introduction of chemical substituents.

The inventors of the present invention have found that the carbon-containing residue produced in fullerene production or carbon nanostructures production has valuable properties after functionalization. In particular, the examples show that rubber/carbon black/silane compounds produced with the inventively functionalized residue, unlike rubber compounds produced with known carbon blacks, exhibit behaviour typical of mixtures with low rolling loss.

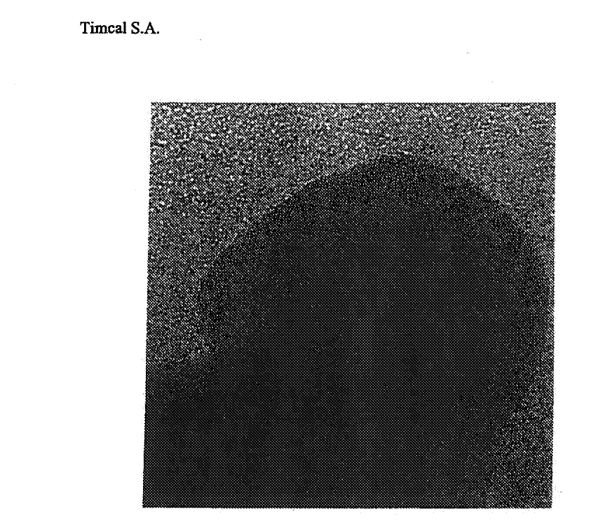

FIG. 1 shows a transmission electron micrograph of a fullerene residue obtained from a plasma process. We clearly see the total covering of the carbon black surface via fullerene-type carbon layers. These fullerene structures are extremely probably obtained via the condensation of fullerenes, fullerene precursors or fullerene condensates during or after the quenching phase.

When compared with normal carbon black,

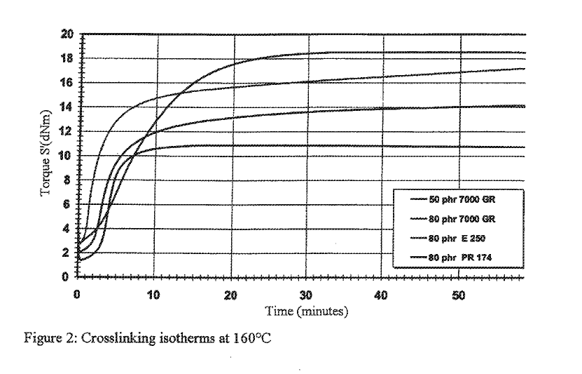

FIG. 2 shows a graph which describes the development of the crosslinking isotherms of the mixtures over time. The functionalized fullerene carbon black clearly shows the strong interaction between carbon black and polymer.

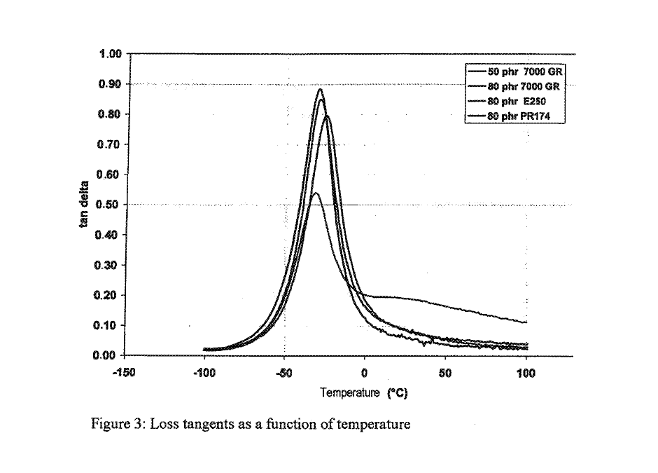

FIG. 3 shows the dependency of tan delta on temperature for various rubber compounds produced. The mixture which comprises the fullerene carbon black shows behaviour identical with that of the mixtures based on silica. The reference carbon black shows the typical behaviour of carbon black, high tan delta values at high temperatures and low tan delta at low temperatures.

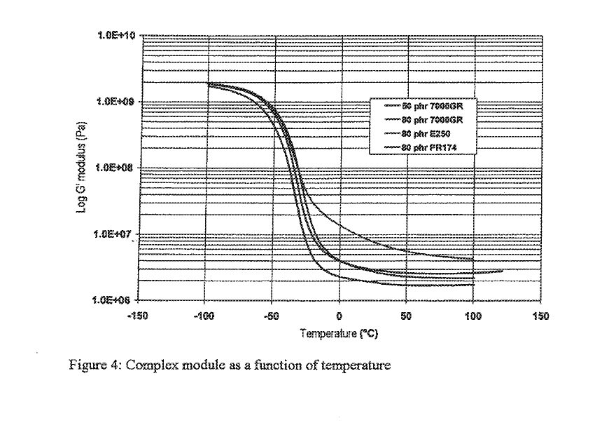

FIG. 4 shows the modulus as a function of temperature. Here again, we see full overlap with the results achieved using the silica mixtures.

Some expressions will be defined below in the way in which they are intended to be understood in the context of the invention that follows.

“Carbon-containing residue from fullerene production and carbon-nanostructures production” means a residue which comprises a substantial proportion of fullerene-type nanostructures. The proportion of fullerene-type carbon compounds is determined via the presence of 5- or 6-membered carbon rings which lead to curved layers of carbon on the carbon black surface. The proportion of fullerene-type carbon nanostructures here is usually approximately 100%, but can be less. The decisive factor is the requirement to permit functionalization which brings about a significant change in the properties of the carbon black. The proportion is preferably from 80% to 100%. This preferred proportion can change with the application, however.

### DETAILED DESCRIPTION OF THE INVENTION

In principle, any of the known processes for fullerene production and/or carbon-nanostructures production is suitable for obtaining the carbon-containing residue. Furnace blacks or carbon blacks from other processes are also suitable as long as the fullerene-type residues on the surface are sufficient.

According to one preferred embodiment, the carbon-containing residue is obtained via ablation of a carbon electrode by means of an electric arc, a laser, or solar energy. A process described for electric arc ablation is obtainable from Journet, C. et al., Nature 388 (1997), 756. A process suitable for laser ablation of carbon and production of a carbon-containing residue is described in Thess, A. et al., Science 273 (1996), 483. A process suitable for production of carbon-containing residue via chemical vapour deposition using hydrocarbons is described in Ivanov et al., Chem Phys. Lett. 223, 329 (1994). A production process using plasma technology is described in Taiwanese Patent Application No. 93107706. A suitable solar energy process for production of a carbon-containing residue is described in Fields et al., U.S. Pat. No. 6,077,401.

The carbon-containing residue can be obtained via incomplete combustion of hydrocarbons. By way of way of example, fullerene production has been observed in flames derived from premixed benzene/acetylene (Baum et al., Ber. Bunsenges. Phys. Chem. 96 (1992), 841-847). Other examples of hydrocarbons suitable for combustion for the production of a carbon-containing residue are ethylene, toluene, propylene, butylene, naphthalene or other polycyclic aromatic hydrocarbons, in particular petroleum, heavy oil and tar, and these can likewise be used. It is also possible to use materials which are derived from carbon, from carrageen and from biomass and which mainly comprise hydrocarbons but which can also comprise other elements, such as nitrogen, sulphur and oxygen. U.S. Pat. No. 5,985,232 describes a particularly preferred process for combustion of hydrocarbons.

According to another embodiment, the carbon-containing residue can be obtained via treatment of carbon powder in a thermal plasma alongside fullerenes. As an alternative, the carbon-containing residue can be obtained via recondensation of carbon in an inert or to some extent inert atmosphere.

By way of example, PCT/EP94/03211 describes a process for conversion of carbon in a plasma gas. Fullerenes, and also carbon nanotubes, can likewise be produced via this process.

The carbon-containing residue is preferably produced via the following steps, preferably in this sequence:
A plasma is generated with electrical energy.
A carbon precursor and/or one or more catalysts and a carrier plasma gas are introduced into a reaction zone. This reaction zone is, if appropriate, in an airtight vessel that withstands high temperatures.
The carbon precursor is to some extent vaporized at very high temperatures in this vessel, preferably at a temperature of 4000° C. or higher.
The carrier plasma gas, the vaporized carbon precursor and the catalyst are passed through a nozzle whose diameter narrows, widens, or else remains constant in the direction of the plasma gas flow.
The carrier plasma gas, the vaporized carbon precursor and the catalyst are passed through the nozzle into a quenching zone for nucleation, growth and quenching. This quenching zone is operated with flow conditions produced via aerodynamic and electromagnetic forces, so as to prevent any noticeable return of starting material or products from the quenching zone into the reaction zone.
The gas temperature in the quenching zone is controlled at from about 4000° C. in the upper part of this zone to about 800° C. in the lower part of this zone.
The carbon precursor used can be a solid carbon material which involves one or more of the following materials: carbon black, acetylene black, thermal black, graphite, coke, plasma carbon nanostructures, pyrolitic carbon, carbon aerogel, activated carbon or any desired other solid carbon material.
As an alternative, the carbon precursor used can be a hydrocarbon, preferably composed of one or more of the following: methane, ethane, ethylene, acetylene, propane, propylene, heavy oil, waste oil, or of pyrolysis fuel oil or of any other desired liquid carbon material. The carbon precursor can also be any organic molecule, for example vegetable fats, such as rapeseed oil.
The gas which produces a carbon precursor and/or produces the plasma involves and is composed of one or more of the following gases: hydrogen, nitrogen, argon, helium, or any desired other pure gas without carbon affinity, preferably oxygen-free.

With respect to other process variants, reference is made to WO 04/083119, the disclosure content of which is incorporated herein by way of reference.

The carbon is particularly preferably carbon black, graphite, another carbon allotrope or a mixture thereof.

According to the invention, the carbon-containing residue obtained during fullerene production and/or during carbon-nanostructures production is functionalized via introduction of chemical substituents. The functionalization reaction can be carried out during or after the production process.

The functionalization reactions here involve one or more of the following reactions:
Hydroxylation of the residue, preferably via an oxidant, the oxidant particularly preferably being potassium permanganate.
Reaction of the residue with ammonia, obtaining amino groups.
Reaction of the residue with alkyl- or arylamines.
Reaction of the residue with ozone, forming ozonides and subsequently forming carbonyl compounds.
Treatment of the residue with a halogenating agent, the halogenating agent preferably being chlorine or bromine.
Subjection of the residue to a cycloaddition reaction.
Subjection of the residue to a Grignard reaction.
Hydrogenation of the residue.
Subjection of the residue to an electrochemical reaction.
Subjection of the residue to a Diels-Alder reaction.
Formation of donor-acceptor molecule complexes.
Other functionalization reactions suitable alongside the above-mentioned reactions are any of those known from the prior art in connection with fullerenes.

Another aspect of the present invention provides the functionalized carbon-containing residue obtainable via the inventive process.

The functionalized carbon-containing residue is suitable as a hydroxylating agent.

The functionalized carbon-containing residue is moreover suitable as a wetting agent in aqueous systems.

Another application of the functionalized carbon-containing residue consists in the reaction using silanes. The behaviour of the inventively functionalized residue is similar to that of silica in rubber compounds. As is apparent from the example, the residue exhibits an inversion of the loss tangent in the temperature range from −30° C. to 100° C. when used in rubber compounds. This property permits use in tyre treads, where better adhesion at low temperatures and reduced rolling resistance at relatively high temperatures is desired.

Another application of the functionalized carbon-containing residue consists in a means for modification via tether-directed remote functionalization. This method can be used to produce rotaxanes, catenanes, ion sensors and porphyrine conjugates, these being obtainable only with difficulty by other methods.

The inventive functionalized carbon-containing residue can moreover be used for condensation reactions of amines using organic acids.

Another use of the functionalized carbon-containing residue relates to cycloadducts. The functionalized carbon-containing residue can be used here for the polymerization reaction, for example, of cyclopentadiene.

The examples below illustrate the subject matter of the invention, the intention not being, however, that they restrict the subject matter of the invention, but that the present disclosure directly provides the skilled worker with further embodiments of the present invention.

### EXAMPLES

### Example

Four formulations, of which two are based on silica using respectively 50 and 80 parts, one mixture using the reference carbon black which is used in fullerene production as carbon precursor, and the mixture using the hydroxylated fullerene residue.

| | A | B | C | D |
|---|---|---|---|---|
| Buna VSL 5025-1 | 96.25 | 96.25 | 96.25 | 96.25 |
| Buna CB24 | 30 | 30 | 30 | 30 |
| Ultrasil 7000GR | 50 | 80 | 0 | 0 |
| Ensaco 250 | 0 | 0 | 80 | 0 |
| Hydroxylated fullerene carbon black | 0 | 0 | 0 | 80 |
| (PR174) | | | | |
| Si-69 | 4.5 | 7.1 | 7.1 | 7.1 |
| ZnO | 3 | 3 | 3 | 3 |
| Stearic acid | 1 | 1 | 1 | 1 |
| TMQ | 1 | 1 | 1 | 1 |
| 6PPD | 1 | 1 | 1 | 1 |
| Antilux 654 | 1.5 | 1.5 | 1.5 | 1.5 |
| Plasticizer 450 | 8 | 8 | 8 | 8 |
| Sulphur | 1.5 | 1.5 | 1.5 | 1.5 |
| Vulkacit CZ | 1.5 | 1.5 | 1.5 | 1.5 |
| Vulkacit D | 2 | 2 | 2 | 2 |

### Production of Mixture

The mixtures were produced in four stages in a “Haake Polylab Rheomix 600” test kneader system and on a laboratory roll mill.

Stage 1: Basic mixing stage (test kneader)
Stage 2: Remill stage 1 (test kneader)
Stage 3: Remill stage 2 (test kneader)
Stage 4: Mixing to incorporate sulphur and accelerators (roll mill)

Between the individual stages, the sheet composed of the mixture was stored at room temperature for 24 h. The batch temperatures reached in the first 3 stages were from 150 to 160° C. The parameters for production of the mixture are as follows:

| | |
|---|---|
| Stage 1 | |
| Kneader fill level: | 70% |
| Prior temperature setting: | 140° C. |
| Rotor rotation rate: | 50 rpm |
| Mixing time: | 10 minutes |
| Stage 2 | |
| Kneader fill level: | 70% |
| Prior temperature setting: | 140° C. |
| Rotor rotation rate: | 50 rpm |
| Mixing time: | 8-10 minutes |
| Stage 3 | |
| Kneader fill level: | 70% |
| Prior temperature setting: | 140° C. |
| Rotor rotation rate: | 100 rpm |
| Mixing time: | 8-10 minutes |
| Stage 4 | |
| Roll temperature: | cooled |
| Roll rotation rate: | 16:20 rpm |
| Mixing time: | 7 minutes |

### Vulcanization

Test sheets of thickness 2 mm were vulcanized at 160° C. Vulcanization time was t₉₀+2 minutes.

### Results

Rheometer data at 160° C.

| | A | B | C | D |
|---|---|---|---|---|
| Min. torque | 1.39 | 2.07 | 2.73 | 2.8 |
| Max. torque | 10.89 | 14.15 | 17.24 | 18.54 |
| Delta torque | 9.5 | 12.08 | 14.51 | 15.74 |
| Time to 90% | 6.92 | 17.17 | 22.5 | 17.38 |

The mixture based on the hydroxylated fullerene residue shows the same picture in FIG. 3 as the silica mixture. In comparison with the reference carbon black, we observe a spectacular increase in the loss tangent at low temperatures and a noticeably lower tangent at relatively high temperatures.

1. Process for further processing of a carbon-containing residue derived from fullerene production or from carbon-nanostructures production, comprising functionalizing the carbon-containing residue.

2. Process according to claim 1, where the carbon-containing residue is obtained via ablation of a carbon electrode by means of an electric arc, a laser or solar energy.

3. Process according to claim 1, where the carbon-containing residue is obtained via incomplete combustion of hydrocarbons.

4. Process according to claim 1, where the carbon-containing residue is obtained via treatment of carbon powder in a thermal plasma.

5. Process according to claim 1, where the carbon-containing residue is obtained via recondensation of gaseous carbon in an inert or to some extent inert atmosphere.

6. Process according to claim 4, where the carbon powder is carbon black, graphite, another carbon allotrope or a mixture thereof.

7. Process according to claim 1, where functionalizing the carbon-containing residue comprises hydroxylating the residue.

8. Process according to claim 7, where the hydroxylation is undertaken by means of an oxidant.

9. Process according to claim 8, where the oxidant is potassium permanganate.

10. Process according to claim 1, where functionalizing the carbon-containing residue comprises reacting the residue with ammonia.

11. Process according to claim 1, where functionalizing the carbon-containing residue comprises reacting the residue with alkyl- or arylamines.

12. Process according to claim 1, where functionalizing the carbon-containing residue comprises reacting the residue with ozone.

13. Process according to claim 1, where functionalizing the carbon-containing residue comprises reacting the residue with a halogenating agent.

14. Process according to claim 13, where the halogenating agent is chlorine or bromine.

15. Process according to claim 1, where functionalizing the carbon-containing residue comprises subjecting the residue to a cycloaddition reaction.

16. Process according to claim 1, where functionalizing the carbon-containing residue comprises subjecting the residue to a Grignard reaction.

17. Process according to claim 1, where functionalizing the carbon-containing residue comprises hydrogenating the residue.

18. Process according to claim 1, where functionalizing the carbon-containing residue comprises subjecting the residue to an electrochemical reaction.

19. Process according to claim 1, where functionalizing the carbon-containing residue comprises subjecting the residue to a Diels-Alder reaction.

20. Process according to claim 1, where functionalizing the carbon-containing residue comprises forming donor-acceptor molecule complexes.

21. (canceled)

22. Functionalized carbon-containing residue obtainable by the process of claim 1.

23. Process of claim 1 further comprising using the functionalized carbon-containing residue as a hydroxylating agent.

24. Process of claim 1 further comprising using the functionalized carbon-containing residue as a wetting agent in aqueous systems.

25. Process of claim 1 further comprising using the functionalized carbon-containing residue as an additive in rubber compounds.

26. Process of claim 1 further comprising using the functionalized carbon-containing residue for tether-directed remote functionalization.

27. Process of claim 1 further comprising using the functionalized carbon-containing residue for the condensation reaction of amines using organic acids.

28. Process of claim 1 further comprising using the functionalized carbon-containing residue in a cycloaddition reaction.

29. Process of claim 1 wherein the functionalizing of said carbon-containing residue occurs after the fullerene production or the carbon-nanostructures production.

30. Process of claim 1 wherein the functionalizing of said carbon-containing residue occurs during the fullerene production or the carbon-nanostructures production.
